SEQUENCE LISTING (16 sequences)

SEQ ID NO: 1 - DNA, 21 bp, Artificial Sequence
  Synthetic PCK9 5′ primer
tggctgcatgacattgcttct

SEQ ID NO: 2 - DNA, 20 bp, Artificial Sequence
  Synthetic PSK9 3′ primer
gcactggagaaccacacagg

SEQ ID NO: 3 - DNA, 17 bp, Artificial Sequence
  Synthetic SREBP1a 5′ primer
gcgccatggacgagctg

SEQ ID NO: 4 - DNA, 17 bp, Artificial Sequence
  Synthetic SREBP1a 3′ primer
ttggcacctgggctgct

SEQ ID NO: 5 - DNA, 20 bp, Artificial Sequence
  Synthetic SREBP1c 5′ primer
ggagccatggattgcacatt

SEQ ID NO: 6 - DNA, 20 bp, Artificial Sequence
  Synthetic SREBP1a 3′ primer
gcttccagagaggaggccag

SEQ ID NO: 7 - DNA, 21 bp, Artificial Sequence
  Synthetic SREBP total 5′ primer
gctccagctcatcaacaacca

SEQ ID NO: 8 - DNA, 21 bp, Artificial Sequence
  Synthetic SREBP total 3′ primer
caggaaggcttccagagagga

SEQ ID NO: 9 - DNA, 20 bp, Artificial Sequence
  Synthetic SREBP2 5′ primer
cccttgacttccttgctgca

SEQ ID NO: 10 - DNA, 19 bp, Artificial Sequence
  Synthetic SREBP2 3′ primer
gcgtgagtgtgggcgaatc

SEQ ID NO: 11 - DNA, 21 bp, Artificial Sequence
  Synthetic HMGCoA Reducatse 5′ primer
gattctggcagtcagtgggaa

SEQ ID NO: 12 - DNA, 20 bp, Artificial Sequence
  Synthetic HMGCoA Reducatse 3′ primer
gttgtagccgcctatgctcc

SEQ ID NO: 13 - DNA, 25 bp, Artificial Sequence
  Synthetic GAPDH  5′ primer
atgttccagtatgactccactcacg

SEQ ID NO: 14 - DNA, 25 bp, Artificial Sequence
  Synthetic GAPDH  3′ primer
gaagacaccagtagactccacgaca

SEQ ID NO: 15 - DNA, 30 bp, Artificial Sequence
  Synthetic  pcDNA3-hPCSK9-55KDa 5′ primer
caggccagcaagtgtgacagtcatggcacc

SEQ ID NO: 16 - DNA, 18 bp, Artificial Sequence
  Synthetic pcDNA3-hPCSK9-55KDa 3′ primer
ctgcgcacgggcgcccgc

(19) United States
(12) Patent Application Publication
(10) Pub. No.: US 2022/0143185 A1
(43) Pub. Date: May 12, 2022

(54) ORALLY ACTIVE SMALL MOLECULE INHIBITOR OF PAI-1

(71) Applicants: Northwestern University, Evanston, IL (US); Renascience, Inc., Tokyo (JP)

(72) Inventors: Douglas E. Vaughan, Chicago, IL (US); Joshua A. Levine, Chicago, IL (US); Toshio Miyata, Tokyo (JP)

(21) Appl. No.: 17/441,484

(22) PCT Filed: Mar. 20, 2020
(86) PCT No.: PCT/US2020/023936

§ 371 Date: Sep. 21, 2021

Related U.S. Application Data
(60) Provisional application No. 62/822,471, filed on Mar. 22, 2019.

(51) Int. Cl. A61K 45/06; A61P 3/06

(52) U.S. Cl. CPC A61K 45/06; A61P 3/06

(57) ABSTRACT

Methods of treating a metabolic disorder comprising orally administering to the patient a small molecule inhibitor of plasminogen activator inhibitor 1 (PAI-1) are described. The treatment with the PAI-1 inhibitor changes at least one metabolic measurement of the patient such as fasting glucose, fasting plasma insulin and fasting plasma LDL when compared to the metabolic measurement of the patient in an untreated condition. The metabolic disorder treated by the methods may include obesity, type 2 diabetes mellitus, nonalcoholic fatty liver disease, non-alcoholic steatohepatitis, hyperlipidemia, metabolic syndrome, and cardiovascular disease, hypercholesterolemia, familial hypercholesterolemia, or elevated LDL.

### CROSS-REFERENCE TO RELATED APPLICATIONS

The present application is a National Stage Entry of PCT International Patent Application No. PCT/US2020/023936 entitled “ORALLY ACTIVE SMALL MOLECULE INHIBITOR OF PAI-1,” filed on Mar. 20, 2020, which claims priority to and the benefit of U.S. Provisional Application No. 62/822,471, filed Mar. 22, 2019, the disclosures of which are all hereby incorporated herein by reference in their entireties for all purposes.

### SEQUENCE LISTING

The instant application contains a Sequence Listing which has been submitted electronically in ASCII format and is hereby incorporated by reference in its entirety. Said ASCII copy, created on Mar. 20, 2019, is named 47460-00_ST25.txt and is 3469 bytes in size.

### BACKGROUND

The obesity-associated metabolic syndrome affects a growing percentage of the American population and increases the risk of cardiovascular disease (CVD) and mortality. The diagnosis of the metabolic syndrome is based on the presence of central obesity, hypertension, dyslipidemia, and insulin resistance.

Hyperlipidemia is a major risk factor for the development of cardiovascular disease. A multitude of literature has shown that lowering low density lipoprotein (LDL) cholesterol with statin therapy improves morbidity and mortality. However, there are many patients who do not achieve target LDL goals with statin therapy alone. PCSK9 inhibitors, when administered with maximum statin therapy, have been shown to further reduce cardiovascular morbidity and mortality in addition to the reduction from the statin therapy alone. The FDA has recently approved the use of therapeutic monoclonal antibodies to PCSK9, and these agents reduce cardiovascular events and mortality in patients on maximum doses of statin therapy. However, PCSK9 immunotherapeutics are expensive and usage is limited to patients with familial hypercholesterolemia or to patients with known cardiovascular disease and elevated LDL.

In mammals, PAI-1 serves as the primary rapid acting inhibitor of tissue-type plasminogen activator (t-PA) and urokinase-type plasminogen activator (uPA). Numerous studies have identified strong statistical correlations between plasma levels of PAI-1 and each of the individual components of the metabolic syndrome. Plasma PAI-1 levels increase exponentially as the number of elements of the metabolic syndrome increase linearly. Plasma PAI-1 levels strongly correlate with body mass index (BMI), and predict the development of the metabolic syndrome and type 2 diabetes mellitus (T2DM). Similar to humans, plasma PAI-1 levels are substantially increased in murine models of obesity and T2DM. Mice with genetic deficiency of PAI-1 are resistant to diet-induced obesity, hepatic steatosis and insulin resistance. Furthermore, pharmacologic inhibition of PAI-1 with an orally active small molecule inhibitor preserves insulin sensitivity and prevents hepatic steatosis in mice. These links between PAI-1 and insulin sensitivity have recently been confirmed in humans. A rare loss-of-function mutation in the gene that codes for PAI-1 (SERPINE1) has been identified in a kindred of Old-Order Amish of northeastern Indiana. Heterozygous carriers of the mutation have lower fasting insulin levels and are protected from the development of T2DM relative to unaffected members of the kindred.

Plasminogen Activator Inhibitor-1 inhibitors have been described in, for example, in U.S. Published Application US 2014/0296256A1 to Toshio Miyata et al, the disclosure of which is hereby incorporated by reference.

### SUMMARY

Clinical features and biomarkers have been linked to obesity and the metabolic syndrome, including increased plasma levels of the serine protease inhibitor plasminogen activator inhibitor-1 (PAI-1). As now summarized, pharmacologic inhibition of PAI-1 results in a significant reduction in plasma cholesterol mediated by direct effects on the production of proprotein convertase subtilisin/kexin type 9 (PCSK9), a regulator of plasma cholesterol levels. This newly discovered mechanistic relationship allows for the identification of PAI-1 as a singular factor that directly contributes to each of the clinical components of the metabolic syndrome.

Plasminogen activator inhibitor 1 (PAI-1) is a member of the serpin family of proteases that inhibits tissue-type plasminogen activator and urokinase. Elevated levels of plasma PAI-1 are one of the hallmarks of obesity and are predictably elevated in patients with the metabolic syndrome and type 2 diabetes mellitus. Beyond these correlative relationships, increased PAI-1 levels are a predictor of, and a potential contributor to, the development of obesity and diabetes.

Provided are methods of treating a metabolic disorder in a patient comprising: orally administering to the patient an inhibitor of plasminogen activator inhibitor 1 (PAI-1). The treatment with the PAI-1 inhibitor changes at least one metabolic measurement of the patient selected from the group consisting of: fasting glucose, fasting plasma insulin and fasting plasma LDL when compared to the metabolic measurement of the patient in an untreated condition.

The inhibitor of PAI-1 used to treat a metabolic disorder may be a small molecule such as TM5614 or TM5A15 and the metabolic disorder treated by the methods may be at least one of: obesity, type 2 diabetes mellitus, nonalcoholic fatty liver disease, non-alcoholic steatohepatitis, hyperlipidemia, metabolic syndrome, and cardiovascular disease, hypercholesterolemia, familial hypercholesterolemia, or elevated LDL.

Other methods features and/or advantages is, or will become, apparent upon examination of the following figures and detailed description. It is intended that all such additional methods, features, and advantages be included within this description and be protected by the accompanying claims.

### BRIEF DESCRIPTIONS OF DRAWINGS

FIG. 1 is a table of RT-PCR primers.

FIG. 2 is the experimental design showing mice fed a fast food diet for 4 months followed by 2 months of fast food diet with or without TM5614.

FIG. 3 is a hierarchical clustering of expression changes seen by RNA-seq.

FIG. 4 is a volcano plot representation of all gene expression changes.

FIG. 5 is a gene ontology analysis of statistical differentially expressed genes.

FIG. 6 is a gene ontology analysis from RNA-seq.

FIG. 7 is a gene ontology pathway analysis showing decreased gene changes from RNA-seq.

FIG. 8 is a gene ontology pathway analysis showing increased gene changes from RNA-seq.

FIG. 9 demonstrates murine plasma PCSK9 levels.

FIG. 10 demonstrates total cholesterol levels in mouse plasma. Values are express as mean±SEM (n=5), * p<0.05, ** p<0.01.

FIG. 11A-B demonstrates TM5614 reduces the HDL-cholesterol levels in mice fed chow diet. 11A. Cholesterol content profile of the lipoprotein fractions resolved by FPLC from pooled samples (n=5 for control, n=5 for TM5614). 11B. Quantification of cholesterol in each lipoprotein fraction calculated using AUC from the FPLC profile.

FIG. 12A-B demonstrates TM5614 improves glucose tolerance and hepatic steatosis. 12A. Initial weights of mice prior to TM5614 treatment. 12B. Percentage weight change during the period of TM5614 administration.

FIG. 13 Long-term TM5614 treatment leads to a reduction in circulatory cholesterol and PCSK9 in mice fed a HFHS diet. WT C57BL/6J male mice fed HFHS diet with and without TM5614. A. PAI-1 activity levels (ng/mL).

FIG. 14 A-B demonstrates long-term TM5614 treatment leads to a reduction in circulatory cholesterol and PCSK9 in mice fed a HFHS diet. WT C57BL/6J male mice fed HFHS diet with and without TM5614. 14A. Cholesterol distribution over the lipoprotein fractions resolved by FPLC from pooled samples (n=2 for HFHS, n=3 for HFHS+TM5614) (2-way ANOVA p=0.03). 14B. Quantification of cholesterol in each lipoprotein fraction calculated using area under the curve from the FPLC profile.

FIG. 15A-C demonstrates long-term TM5614 treatment leads to a reduction in circulatory cholesterol and PCSK9 in mice fed a HFHS diet. WT C57BL/6J male mice fed HFHS diet with and without TM5614. 15A. Hepatic mRNA expression of cholesterol regulatory pathway nodes. 15B. Plasma PCSK9 levels (ng/mL). 15C. LDLR protein levels from liver lysates (ng LDLR/mg Liver Protein). Values are express as mean±SEM (n=7), * p<0.05, ** p<0.01, *** p<0.001.

FIG. 16 A-C demonstrates RT-PCR quantification of mRNA changes with TM5614 in mice FFD fed mice. Note most of the changes are in the cholesterol synthesis and regulation pathway.

FIG. 17A-C demonstrates TM5614 improves glucose tolerance and hepatic steatosis 17A. Intraperitoneal glucose tolerance test. P value represents a 2 way ANOVA. 17B. Hematoxylin and Eosin staining of liver. 17C. Quantification of triglyceride content. Values are expressed as mean±SEM (n=6-7), ** p<0.01.

FIG. 18A-G demonstrates PAI-1 heterozygosity is associated with lower PCSK9 and cholesterol profiles. 20-week-old wild-type or PAI1+/−C57BL/6J male mice fed HFHS diet were challenged with the acute exposure to a PAI-1 inhibitor, TM5614 given orally (20 mg/kg/day) for ten days. At the endpoint murine hepatic mRNA and plasma were analyzed. 18A. Plasma PAI-1 levels. 18B. Cholesterol content profile of the lipoprotein fractions resolved by FPLC from pooled samples (n=6 for WT, n=7 for PAI1+/−). 18C. Plasma total cholesterol 18D. Hepatic PCSK9 mRNA expression normalized to GAPDH. 18E. Plasma PCSK9 levels (ng/mL). 18F. Hepatic PCSK9 mRNA expression normalized to GAPDH across all mouse cohorts. 18G. Plasma PCSK9 levels across all mouse cohorts. Values are expressed as mean±SEM (n=6-7), * p<0.05.

FIG. 19A-E demonstrates PCSK9 is reduced in humans carrying a mutation in SERPINE1, the gene that encodes for PAI-1. Characterization of the Amish cohort human plasma (19A. PAI-1 levels, 19B. PCSK9 levels, and 19C. Correlation between plasma PAI-1 vs. plasma PCSK9 levels) (values are express as mean±SEM (n=7-17). 19D. Relationship between PCSK9 and PAI-1 levels in a separate cohort of HFpEF patients, values are express as mean±SEM (n=147). 19E. PAI-1 levels in human plasma from a cohort of patients with hyperlipidemia before and during treatment with a PCSK9 inhibitor, values are express as mean±SEM (n=28).

FIG. 20 demonstrates PCSK9 levels increase after administration of a PCSK9 monoclonal antibodies. Human plasma PCSK9 levels from before and after administration with a monoclonal antibody against PCSK9.

FIG. 21A-C demonstrates PCSK9 characterization in TM5614 treated PCSK9-HEK293T cells. 21A. HEK293T cells were transfected with PCSK9 (62 kDa) and were incubated with TM5614 (10 μM) or vehicle (DMSO<0.5%) for 48 h. Cell extracts and media were resolved by SDS-PAGE and western blotted for PCSK9 and actin. PNGase F treated cell extracts and media were run along the control samples to confirm the glycosylated form of the upper band of 62 kDa form. Bands corresponding to PCSK9 isoforms Pro-PCSK9, glycosylated and non-glycosylated PCSK9 62 kDa forms, PCSK9 prodomain and actin are highlighted. 21B. Quantification of the different PCSK9 bands (Pro-PCSK9, total PCSK9-62 and glycosylated and non-glycosylated PCSK9 62 kDa forms), normalized to actin, and plotted as relative change to control. 21C. HepG2 cell extracts were resolved by SDS-PAGE and western blotted for LDLR and actin. LDLR levels after treating cells with TM5614 (10 μM) or vehicle (DMSO<0.5%) for 48h. 21B. Quantification of LDLR normalized to actin expression are plotted to the right of the representative blot image. Values are express as mean±SEM (n>3), ** p<0.01.*** p<0.001.

FIG. 22 demonstrates In vitro LDLR characterization in cells treated with TM5614 or overexpressing uPA. HepG2 cell extracts were resolved by SDS-PAGE and western blotted for LDLR and actin. LDLR levels after treating cells with TM5614 (10 μM) or vehicle (DMSO<0.5%) for 48h. B. Quantification of LDLR normalized to actin expression are plotted to the right of the representative blot image. Values are express as mean±SEM (n>3), ** p<0.01.

FIG. 23 demonstrates Co-treatment of TM5614 and tunicamycin in PCSK9-HEK293T cells. HEK293T cells were transfected with PCSK9 (62 kDa) and were incubated with vehicle (DMSO<0.5%), tunicamycin (4 μg/mL), TM5614 (10 or the combination of the two (2.5 or 4 μg/mL of tunicamycin+10 μM TM5614) for 48 h. Cell extracts and media were resolved by SDS-PAGE and western blotted for PCSK9. Bands corresponding to PCSK9 isoforms Pro-PCSK9, glycosylated and non-glycosylated PCSK9 62 kDa forms and PCSK9 prodomain are highlighted.

FIG. 24 demonstrates PCSK9 characterization in TM5614 treated HepG2 cells. A. Cell extracts or culture media of WT or PCSK9-overexpressing HepG2 cells (transient by overexpression of PCSK9-pcDNA3 or stable hPCSK9tg-HepG2 cells) incubated with TM5614 (10 μM) or vehicle (DMSO<0.5%) (48 h) were resolved by SDS-PAGE and western blotted for PCSK9 and actin. Bands corresponding to PCSK9 isoforms glycosylated and non-glycosylated Pro-PCSK9, glycosylated and non-glycosylated PCSK9 62 kDa forms, PCSK9 prodomain and actin are highlighted.

FIG. 25A-B demonstrate In vitro LDLR characterization in cells treated with TM5614 or overexpressing uPA. 25A: HepG2 cell extracts were resolved by SDS-PAGE and western blotted for LDLR and actin. LDLR levels after treating cells with TM5614 (10 μM) or vehicle (DMSO<0.5%) for 48h. 25B. Quantification of LDLR normalized to actin expression are plotted to the right of the representative blot image. Values are express as mean±SEM (n>3), ** p<0.01.

FIG. 26A-B demonstrates an in vitro assay showing PCSK9 can be cleaved by uPA. This cleavage is reduced with the addition of PAI-1. Inhibition of PAI-1 with TM5A15 rescues this cleavage. 26A is a Western Blot for PCSK9. 26B is a silver stain.

### DETAILED DESCRIPTION

Herein described, PAI-1 is the critical mechanistic factor that links all the major components of the metabolic syndrome. PAI-1 is herein demonstrated to be a critical regulator of lipid homeostasis, thereby adding the fourth and final element of metabolic syndrome. Specifically PAI-1 is associated with hyperlipidemia and PCSK9. Inhibition of PAI-1 with TM5614, a PAI-1 inhibitor, leads to a decrease in PCSK9 at both the transcriptional and protein level. PAI-1 also regulates the transcription of other critical factors involved in lipid biosynthesis, including SREBP1a, SREBP1c, SREBP2, and HMG-CoA reductase. These alterations directly lead to a reduction in total cholesterol, LDL-C, and HDL-C. Taken together, these data demonstrate that PAI-1 not only regulates glucose homeostasis and hepatic steatosis, but importantly, plays a central role in lipid metabolism. Thus, PAI-1 can now be singularly recognized as a mechanistic contributor to all of the essential clinical elements that comprise the metabolic syndrome in humans, including hypertension, insulin resistance, central obesity, and now, dyslipidemia.

As PAI-1 has a role in the expression of genes responsible for lipid metabolism, the treatment described herein involving a PAI-1 inhibitor may decrease the total plasma cholesterol, LDL, or HDL concentration or a combination of these. The effects of inhibiting PAI-1 on hepatic expression of PCSK9 and on plasma PCSK9 concentrations are associated with significant reductions in serum cholesterol. Thus effective treatments may cause either inhibition of PCSK9 or a decrease in levels of PCSK9 in the blood, or both. The result of the inhibition or decrease of PCSK9 is a decrease the fasting plasma LDL in a subject.

PAI-1 inhibition has a robust effect to increase the expression of FGF21. FGF21 reduces cholesterol synthesis and attenuates hypercholesterolemia by suppressing the hepatic expression of SREBP2.

Oral administration of TM5614 effectively reduced hepatic PCSK9 mRNA by over 80% with a corresponding 55% decrease in plasma PCSK9 levels. These effects were paralleled by a significant reduction in plasma HDL-C and LDL-C. The observed decrease in both HDL-C and LDL-C reported here is consistent with other studies in mice using monoclonal antibodies against PCSK9. Importantly, other genes involved in lipid synthesis and metabolism were also downregulated in this experimental model of diet-induced dyslipidemia, including SREBP1/2 and HMG-CoA reductase.

Apart from the effects of PAI-1 inhibition at the transcriptional level, we also found that TM5614 had additional post-translational effects on PCSK9, specifically involving the selective degradation of the non-glycosylated form of the protein. PAI-1 may regulate the ratio of glycosylated and non-glycosylated PCSK9 through a furin-dependent mechanism.

A genetic deficiency of PAI-1 is also associated with a reduction of plasma PCSK9 levels in humans. Herein described is a robust correlation between plasma levels of PAI-1 and PCSK9 in patients that do not have genetic PAI-1 deficiency.

Pharmacological inhibition of PAI-1 prevents hepatic steatosis and reduces serum cholesterol levels through a PCSK9-dependent mechanism. Given that PAI-1 levels are consistently elevated in the millions of patients with the metabolic syndrome, our demonstration of a direct role for PAI-1 in the regulation of lipid metabolism provides further support to the central role of PAI-1 in the pathogenesis of cardiovascular complications associated with the metabolic syndrome, and in the molecular pathogenesis of the metabolic syndrome itself.

In some aspects the invention comprises a method of treating a metabolic disorder in a subject comprising: administering to the subject an inhibitor of plasminogen activator inhibitor 1 (PAI-1). The PAI-1 inhibitor treatment results in a change of at least one metabolic measurement of the subject selected from the group consisting of: fasting glucose level, fasting plasma insulin level, fasting plasma LDL, plasma cholesterol level, plasma HDL, plasma PCSK9 level, plasma plasminogen activator inhibitor 1 (PAI-1) activity, or a combination thereof, when compared to the metabolic measurement of a subject in an untreated condition.

In some aspects, the inhibitor of plasminogen activator inhibitor 1 (PAI-1) is TM5614, TM5A15, or a combination thereof.

In some aspects, the inhibitor of plasminogen activator inhibitor 1 (PAI-1) is administered orally.

In some aspects, the metabolic disorder may comprise obesity, type 2 diabetes mellitus, nonalcoholic fatty liver disease, non-alcoholic steatohepatitis, hyperlipidemia, metabolic syndrome, and cardiovascular disease, hypercholesterolemia, familial hypercholesterolemia, elevated LDL, HDL, or a combination thereof.

In some aspects, the plasminogen activator inhibitor 1 (PAI-1) inhibitor decreases plasma cholesterol by about 25% to about 40%.

In some aspects, the plasminogen activator inhibitor 1 (PAI-1) inhibitor decreases HDL-C by about 30%.

In some aspects, the plasminogen activator inhibitor 1 (PAI-1) inhibitor decreases LDL-C by about 50%.

In some aspects, the plasminogen activator inhibitor 1 (PAI-1) inhibitor reducing PAI-1 activity by about 50%.

In some aspects, the plasminogen activator inhibitor 1 (PAI-1) inhibitor reducing hepatic PCSK9 mRNA expression by about 80%, thereby reducing plasma PCSK9 protein. In some aspects, the plasminogen activator inhibitor 1 (PAI-1) inhibitor effectively reducing plasma PCSK9 protein by about 25% to about 50%.

In some aspects, the plasminogen activator inhibitor 1 (PAI-1) inhibitor effectively reducing plasma non-glycosylated PCSK9 protein.

In some aspects, the method may include co-administration to the subject of a statin and plasminogen activator inhibitor 1 (PAI-1) inhibitor.

In some aspects, the invention provides a method for reducing plasma LDL, HDL, cholesterol, or a combination thereof in a subject. The method may include orally administering to the subject a therapeutically effective amount of a plasminogen activator inhibitor 1 (PAI-1) inhibitor.

In some aspects, the inhibitor of plasminogen activator inhibitor 1 (PAI-1) is TM5614, TM5A15, or a combination thereof.

In some aspects, the method includes co-administration to the subject of a statin and plasminogen activator inhibitor 1 (PAI-1) inhibitor.

In some aspects, the method includes co-administration to the subject of a PCSK9 inhibitor and plasminogen activator inhibitor 1 (PAI-1) inhibitor.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein “levels” refers to a measurable amount of a protein or chemical in a sample. The sample may be a blood, plasma, or tissue sample, for example. A level may be measured in any unit, for example ng/mL or ng/mg, mg/dL, a percent of a total, or any other quantity.

As used herein “activity” refers to any measurable function of a protein. Any assay that can measure the function of an enzyme may be encompassed.

As used herein “metabolic disorder” refers to any obesity-associated metabolic syndrome, obesity, hypertension, dyslipidemia, insulin resistance, diabetes mellitus or the like.

As used herein “treat”, “treating” refers to administering to a subject a PAI-1 inhibitor for the purposes of improving a metabolic measurement.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

As used herein, the term “prevention” refers to all actions that inhibit or delay the development of a target disease. As used herein, the term “prevention” means administering the oxyntomodulin derivative of the present invention to inhibit or delay the development of hyperlipidemia, fatty liver disease or atherosclerosis, which shows an increase in blood total cholesterol and low-density cholesterol levels and a decrease in high-density cholesterol levels.

As used herein, the term “treatment” refers to all actions that alleviate, ameliorate or relieve the symptoms of the disease developed. As used herein, the term “treatment” means administering the inhibitor of PAI-1 of the present invention to alleviate, ameliorate or relieve hyperlipidemia, fatty liver disease or atherosclerosis, which shows an increase in blood total cholesterol and low-density cholesterol levels and a decrease in high-density cholesterol levels.

As used herein, the term “hyperlipidemia” refers to a condition associated with abnormally elevated levels of lipids, such as free cholesterol, cholesterol esters, phospholipids and triglycerides, in blood. Although hyperlipidemia does not show specific symptoms by itself, excessive lipids in blood adhere to the blood vessel walls to reduce the blood vessel size and cause atherosclerosis by inflammatory reactions. For this reason, coronary heart disease, cerebrovascular disease, obstruction of peripheral blood vessels, etc., can occur.

Thus, the pharmaceutical composition of the present invention can be used for the treatment of type 2 diabetes mellitus, nonalcoholic fatty liver disease, non-alcoholic steatohepatitis, hyperlipidemia, metabolic syndrome, and cardiovascular disease, hypercholesterolemia, familial hypercholesterolemia, or elevated LDL or HDL, fatty liver disease or atherosclerosis, but also coronary heart disease, cerebrovascular disease, or obstruction of peripheral blood vessels.

The pharmaceutical composition may further include suitable carriers, excipients and diluents conventionally used in the production of pharmaceutical composition. The pharmaceutical composition may be formulated in the form of oral preparations such as powders, granules, tablets, capsules, suspensions, emulsions, syrups, aerosols or the like, external preparations, suppositories, and sterilized injection solutions according to a conventional method. Specific examples of carriers, excipients and diluents that can be included in the composition may include, but are not limited to, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oil, and the like. In the case of formulation, diluents or excipients such as fillers, extenders, binders, wetting agents, disintegrants, or surfactants, can usually be used.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

Pharmaceutical compositions may be prepared by mixing one or more compounds of the present technology, pharmaceutically acceptable salts thereof, stereoisomers thereof, tautomers thereof, or solvates thereof, with pharmaceutically acceptable carriers, excipients, binders, diluents or the like to prevent and treat disorders associated with the effects of increased plasma and/or hepatic lipid levels. The compounds and compositions described herein may be used to prepare formulations and medicaments that prevent or treat a variety of disorders associated with increased plasma and/or hepatic lipid levels, e.g., hyperlipidemia, hypercholesterolemia, hepatic steatosis, and metabolic syndrome. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co. New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until the elevated plasma or hepatic cholesterol or triglycerides or progression of the disease state is decreased or stopped. The progression of the disease state can be assessed using in vivo imaging, as described, or by taking a tissue sample from a patient and observing the target of interest therein. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of antihyperlipidemia treatment according to the present technology. For example, blood tests to measure total cholesterol as well as triglycerides, LDL and HDL levels are routinely given. Individuals with a total cholesterol level of greater than 200 mg/dL are considered borderline high risk for cardiovascular disease. Those with a total cholesterol level greater than 239 mg/dL are considered to be at high risk. An LDL level of less than 100 mg/dL is considered optimal. LDL levels between 130 to 159 mg/dL are borderline high risk. LDL levels between 160 to 189 mg/dL are at high risk for cardiovascular disease and those individuals with an LDL greater than 190 mg/dL are considered to be at very high risk for cardiovascular disease. Triglyceride levels of less than 150 mg/dL is considered normal. Levels between 150-199 mg/dL are borderline high and levels above 200 mg/dL are considered to put the individual at high risk for cardiovascular disease. Lipid levels can be determined by standard blood lipid profile tests. Effective amounts of the compositions of the present technology will lower elevated lipid levels by at least 10%.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When “only A or B but not both” is intended, then the term “only A or B but not both” will be employed. Thus, use of the term “or” herein is the inclusive, and not the exclusive use. As used in the specification and the claims, the singular forms “a,” “an,” and “the” include the plural. Finally, where the term “about” is used in conjunction with a number, it is intended to include ±10% of the number. For example, “about 10” may mean from 9 to 11. The term wt % is meant to describe a comparison of the weight of one compound to the weight of the whole composition expressed as a percent. It can also be described as wt. %, or (w/w) %. A (w/w) % indicated the amount of a component in relation to the total composition of the traction fluid. Reference to a numeral 5 is equivalent to 5.0.

Certain embodiments are described below in the form of examples. While the embodiments are described in considerable detail, it is not the intention to restrict or in any way limit the scope of the appended claims to such detail, or to any particular embodiment.

### Examples

Materials and Methods

Materials

TM5614 (FW=446.43 g/mol). A 20 mM stock solution was prepared in DMSO and stored at −20° C. In cell culture, TM5614 was added to a final concentration of 10 μM with DMSO<0.5%. For animal feeding experiments, dry TM5614 was mixed into the powdered mouse food which were then formed into pellets given at a dose of 20 mg/kg/day.

Human Cohorts

Plasma samples from members of the Bern Amish Kindred who harbor a frameshift mutation in SERPINE1 (SERPINE1^{+/+} (n=17, 7 males), SERPINE1^{+/-} (n=16, 8 males) and SERPINE1^{-/-} (n=7, 2 males)) were assessed to quantify PCSK9 plasma levels. This study was previously approved by both the Northwestern University and the Indiana Hemophelia and Thrombosis Center Institutional Review Board as previously described.

Plasma samples from hypercholesterolemic patients were obtained from the OHSU Center for Preventive Cardiology Registry and Biorepository, approved by OHSU Institutional Review Board (17329).

Data was also analyzed from a cohort of patients with HFpEF. Patients were enrolled in this prospective observational study from the outpatient HFpEF clinic (for HFpEF patients) and from the outpatient general cardiology clinic or cardiac catheterization laboratory (elective cases) for the control patients. Control patients were deemed eligible if they had 1 or more risk factors for HFpEF (e.g., hypertension, obesity, diabetes, chronic kidney disease) and only if there was no history of heart failure (any type, including those with recovered LVEF). In this analysis, 248 unique plasma proteins (including PAI-1 and PCSK9) were quantified by a multiplex immunoassay (Olink, www.olink.com) using the commercially available panels cardiovascular disease II, IIII, and inflammation. This study was approved by the Northwestern University Institutional Review Board.

Animal Models

Wild-type C57BL/6J male mice (Jackson Laboratories, Bar Harbor, Me.) were fed a standard chow diet and underwent 14/10-hour light/dark cycling with free access to food and water. Animal protocols were approved by the Northwestern University Institutional Animal Care and Use Committee (IACUC). A first cohort of 20-week-old mice was used to evaluate the acute effect of inhibiting PAI-1 with TM5614. Half of the mice were fed standard chow and the other half chow supplemented with TM5614 at a dose of 20 mg/kg/day for ten days. A second cohort of 6-week-old mice was used to evaluate the long-term effects of inhibiting PAI-1 with TM5614 in mice fed a high-fat, high-cholesterol, high-sugar diet (HFHS). The HFHS diet is composed of 40% energy as fat (milk fat, 12% saturated) with 2% cholesterol (AIN-76 Western Diet, Test Diet) and drinking water supplemented with 42 g/l of 55% fructose/45% glucose by weight. Mice were fed HFHS for 4 months to induce obesity. After this period, half of the mice were treated with TM5614 (as described for the acute exposure) for a period of 2 months. At the end of both protocols, blood was collected by retro-orbital bleeding and centrifuged to collect plasma and mice were euthanized by isoflurane followed by cervical dislocation. Mouse livers were flushed with ice-cold saline, a portion of the liver was put into RNA LATER (Cat #76104, Qiagen) prior to RNA extraction. The remainder of the tissue was snap frozen in liquid nitrogen.

RNA Sequencing and Analysis

RNA isolation and sequencing were performed as previously described (37). Briefly, total RNA was extracted from murine livers using an RNA Easy mini kit (Cat #: 74104, Qiagen) and 1 μg of RNA were used for sequencing. RNA libraries were constructed using KAPA mRNA HyperPrep kits (Cat #kk8580, KAPA) according to the manufacturer's instructions. Libraries were quantified using Bioanalyzer (Agilent) and sequenced on an Illumina NextSeq 500 instrument using 75 bp single-end reads. Sequenced reads were aligned to the mm10 reference mouse genome using STAR and differentially expressed RNAs were determined by analyzing with DESeq2 (FDR-adjusted p value <0.05). STAR alignments and DESeq2 were performed though the RNA Express BaseSpace application (Illumina). Morepheus (software.broadinstitute.org/morpheus/) was used to create a hierarchical clustering (heat map) was used to group genes on the basis of similar expression patterns over the samples and a volcano plot was generated to study the differentially expressed genes. The gene list was classified according to gene ontology biological processes. Metascape (http://metascape.org, June 2019) was used for ontology analysis. Promotor motif analysis was performed using HOMER.

Gene Expression

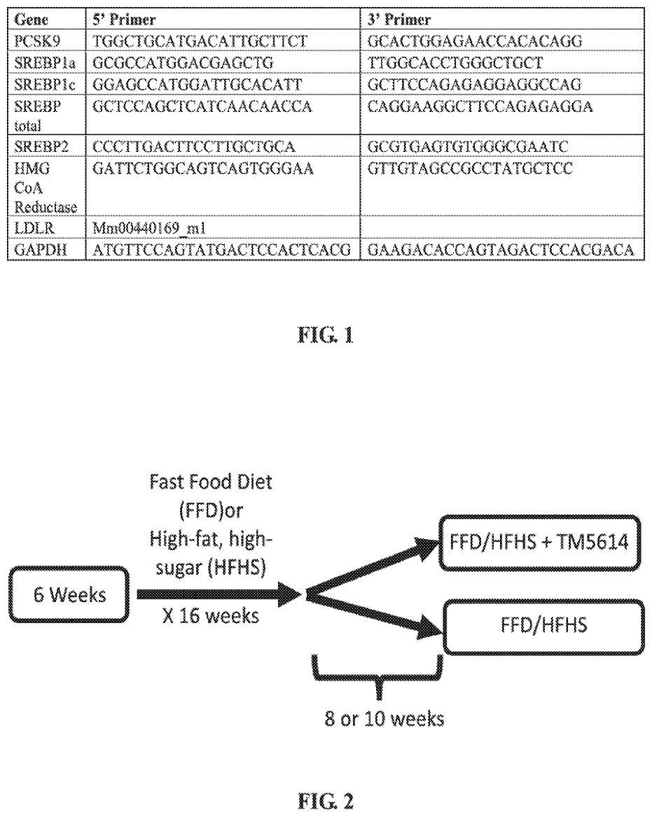

Total RNA was extracted from murine livers using the RNA Easy mini kit (Qiagen, Cat #74104) and complementary DNA was synthesized from 1 μg of mRNA with the qScript cDNA Synthesis kit (Quantabio, Cat #95048), in accordance with the manufacturer's instructions. qRT-PCR was performed on 2 μL of cDNA using either SYBR-Green (Biorad, Cat #1725270) or Taqman primer/probe mixes (Thermo Fisher Scientific). Referring to FIG. 1, a table of primers can be found in the supplementary materials. The reactions were performed following the manufacturer's instructions. GAPDH was employed as a housekeeping gene and fold changes in gene expression were calculated using the standard ΔΔCt method.

Plasma Analysis

Total cholesterol was quantified in the murine plasma using an enzymatic determination (Pointe Scientific, Cat #C7510). The lipoprotein profile of the murine plasma samples was assessed running a fast-phase liquid chromatography (FPLC), in brief 100 μL of plasma (from one animal or a pool of several samples of the same conditions) were loaded to a Superose 6 column (GE HealthCare, Cat #29-0915-96) at a flow rate of 0.5 mL/min in running buffer (0.15M NaCl, 0.01M Na2HPO4 and 1 mM EDTA). Fractions were collected every minute for 41 minutes and analyzed for cholesterol concentrations as described above. Murine plasma PCSK9 levels were determined using an ELISA kit (MBL International, Cat #CY-80781). Murine PAI-1 levels were quantified using an ELISA kit (Molecular Innovations, Cat #MPAIKT-TOT). Murine PAI-1 activity levels were quantified by ELISA (Molecular Innovations, Cat #MPAIKT). Human PAI-1 levels were quantified by ELISA (Molecular Innovations, Cat #HPAIKT-TOT).

Hepatic Protein Quantifications

Hepatic proteins were extracted from liver tissue homogenized with lysis buffer (RIPA Buffer (Sigma-Aldrich, Cat #R0278) supplemented with 1× proteinase inhibitor (Thermo Fisher Scientific, Cat #A32963) and total protein content were performed as described. mLDLR (R&D Systems, Cat #MLDLRO), mPCSK9 (Cat #CY-8078, MBL International), or hPCSK9 (MBL International, Cat #CY-8079) protein levels were quantified from the total extracts (20, 160 and 80 μg, respectively) using ELISA methodology following the manufacturers recommendation.

In Vitro Cellular Studies

Human hepatocellular carcinoma HepG2 (ATCC® HB-8065™) and embryonic kidney HEK293T (ATCC® CRL-1573™) cell lines were routinely grown as previously described (M. D. Senagolage et al., Loss of Transcriptional Repression by BCL6 Confers Insulin Sensitivity in the Setting of Obesity. Cell Rep 25, 3283-3298 e3286 (2018)) at 37° C. in a 5% CO2 humidified atmosphere. 2.5×10⁵ cells were plated. The following day (day 2) media was changed to serum-free media and in some instances, cells were transiently transfected using Fugene 6 (Promega, Cat #E269) following vendor instructions (using 4.5 μL of Fugene per 1.5 μg of DNA plasmid). The expression vectors used were human PCSK9 (pcDNA3-hPCSK9-62 kDa; pcDNA3-hPCSK9-55 kDa generated by inverse PCR (FW-5′-CAGGCCAGCAAGTGTGACAGTCATGGCACC-3′(SEQ ID NO: 15), RV 5′-CTGCGCACGGGCGCCCGC-3′(SEQ ID NO: 16)) from our pcDNA3-hPCSK9-62 kDa), human uPA (pCMV6-XL5-huPA, Origene, SC118493). On day three cells could be treated with 10 μM TM5614 (or vehicle) for 48 h. On day five, cell culture media was collected and stored at −20° C. until analysis. Cells were washed twice with ice-cold phosphate-buffered saline and scraped in 100 μl lysis buffer (same buffer as described for tissue protein extraction). Lysates were incubated on ice for 1 hour (with mixing every 15 minutes) and spun at 18,000×g at 4° C. for 20 minutes, supernatants were collected and quantified by a Lowry assay (DC™ Protein Assay, Cat #Bio-Rad, 5000111) then stored at −20° C. until used. 20 μL of cell culture medium or 40 μg of total extracts were resolved in SDS-PAGE (4-12% or 12% Bis-Tris precast acrylamide gels, Thermo Fischer Scientific) and later transferred to nitrocellulose membranes (GE Healthcare, Cat #10600003) and blot for human PCSK9 (using a rabbit anti-PCSK9 (1:1000 dilution, MBL International, Cat #CY-P1037) as a primary antibody and a goat anti-rabbit (1:15000 dilution, LI-COR Biosciences, Cat #925-32211) as the secondary antibody), LDLR (using a goat anti-LDLR (1:1000 dilution, R&D, Cat #AF2148) as the primary antibody and a donkey anti-goat (1:15000 dilution, LI-COR Biosciences, Cat #925-32214) as the secondary antibody), actin (using a mouse anti-actin (1:2000 dilution, Sigma-Aldrich, Cat #A5441) as the primary antibody and a goat anti-mouse (1:15000 dilution, LI-COR Biosciences, Cat #926-68070) as the secondary antibody), vinculin (using a rabbit anti-vinculin (1:2500 dilution, Abeam, Cat #ab129002) as the primary antibody and a goat anti-rabbit (1:15000 dilution, LI-COR Biosciences, Cat #926-68071) as the secondary antibody). Cell culture media (9 μL) and cell extracts (20 μg) were subjected to N-glycosylation removal with the amidase PNGase F that cleaves between the innermost GlcNAc and the asparagine residue of high mannoses, following vendor instructions (New England Biolabs, Cat #CP0704S) and later subjected to Western blot as described above.

Glucose Tolerance Testing

Intraperitoneal glucose tolerance tests (IPGTT) were performed on mice fasted for 16 hours. Glucose was injected by IP at 2 g/kg of body weight. Blood glucose was obtained from tail veins at multiple time points (0-120 min) and measured using a NovaMax Plus glucometer (Novacares).

Hepatic Triglyceride Content

Hepatic triglyceride levels were measured using an Infinity spectrophotometric assay (Thermo Electron Corporation, Cat #TR22421) following lipid extraction using the Folch method.

Histologic Analysis

Liver was fixed in 10% Phosphate Buffered Formalin (Newcomer Supply, Cat #1090N) and embedded in paraffin. Section were stained with hematoxylin and eosin (H&E) ((Newcomer Supply, Cat #1201 and 1082A).

Statistical Analysis:

The RNA seq data was deposited to GEO, accession #GSE140725. Statistical analyses including unpaired t test, paired t test, linear regression, one-way ANOVA, and two-way ANOVA were performed in Prism version 8 (www.graphpad.com). P values of <0.05 were considered statistically significant.

### Example 1: Protocol Demonstrating the Effects of the Novel PAI-1 Inhibitor TM5614

Referring to FIG. 2, a protocol outline is shown where C57/B6 male mice were fed a fast food (Western Diet+42 g/L of glucose and fructose mixture) or high fat/high sugar diet for 16 weeks (FFD). Mice were then treated with 20 mg/kg of TM5614, a PAI-1 inhibitor for 8 weeks. After 1 week of treatment, the mice exhibited improvement in several metabolic measurements, including fasting glucose (from 131.2 to 92.7 mg/dL, p<0.0001), fasting plasma insulin (from 0.507 to 0.3722 ng/mL, p=0.0278), fasting plasma total cholesterol (from 205 to 196 mg/dL), fasting plasma HDL (from 139 to 134 mg/dL), and fasting plasma LDL (from 48 to 43 mg/dL). Hepatic steatosis was decreased by 35% (p=0.04). After 8 weeks of treatment, there was an improvement in intraperitoneal glucose tolerance testing (2 way ANOVA p=0.03) and a trend in improvement in intraperitoneal insulin tolerance testing. Hepatic steatosis was also decreased. Fasting lipid profile was also reduced after 8 weeks of TM5614 (Total Cholesterol: 354.2 to 228 mg/dL, p=0.0003; HDL: 221 to 148.3 mg/dL, p=0.02; LDL: 86.1 to 44.1, p=0.06).

### Example 2: Acute PAI-1 Inhibition with TM5614 Decreases PCSK9 Expression In Vivo

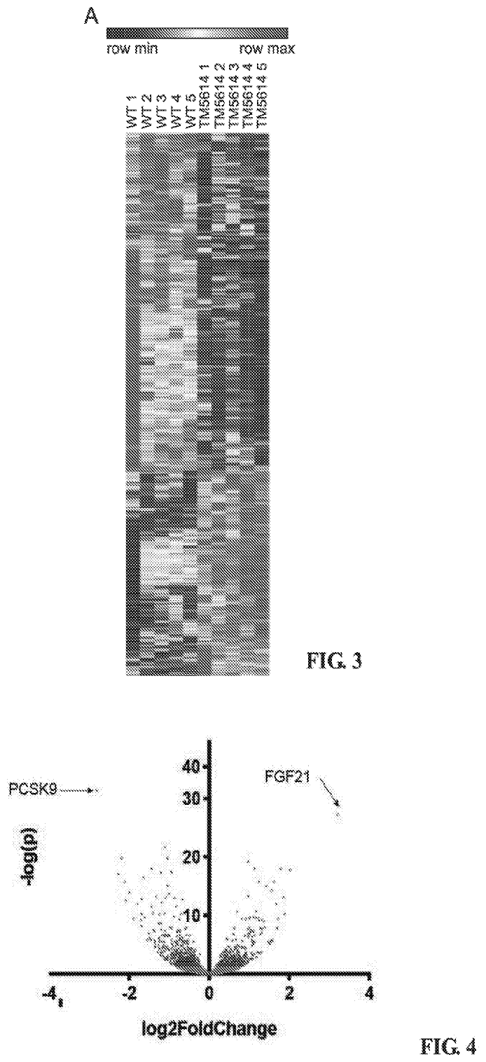

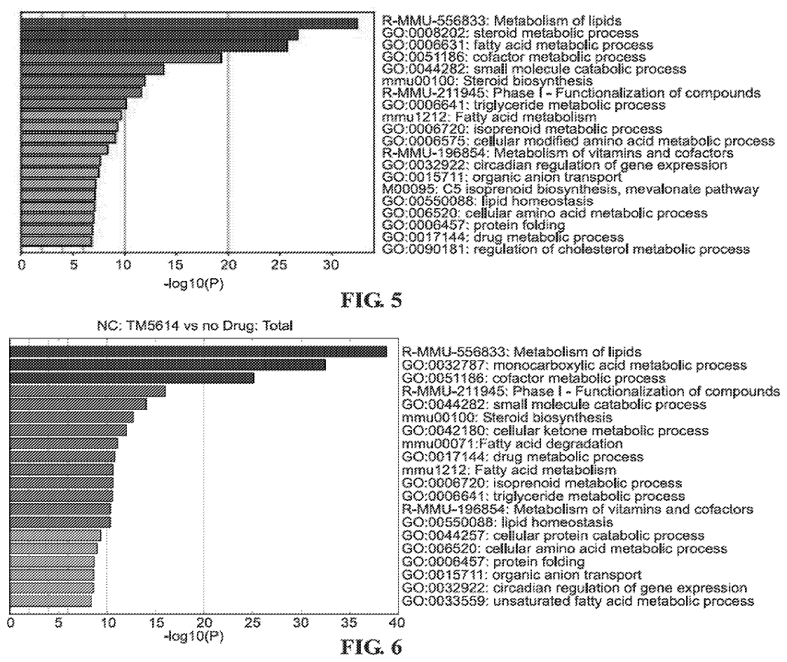

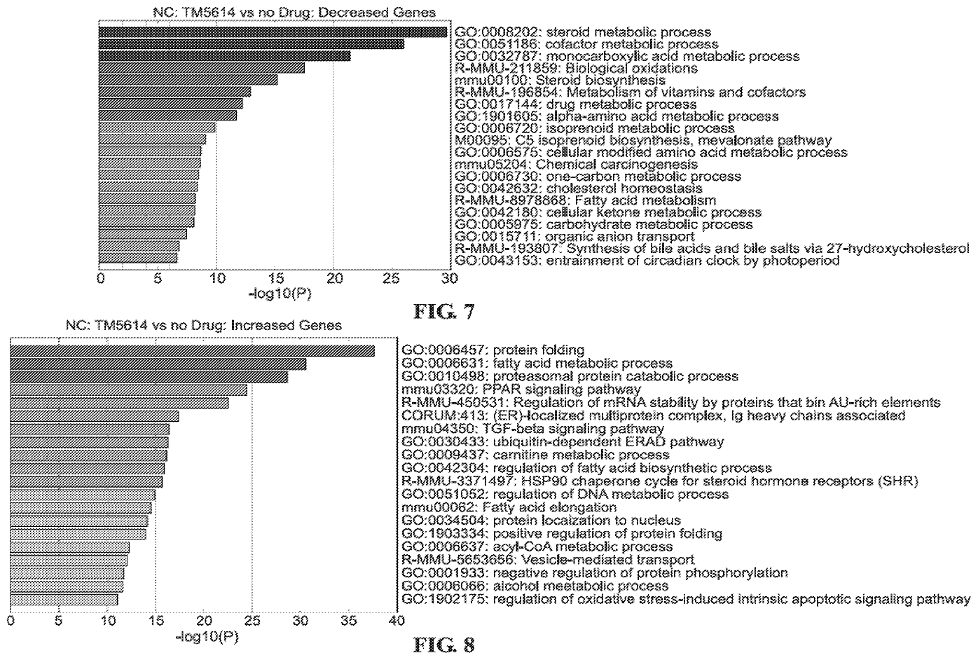

To investigate the mechanistic role of PAI-1 in hepatic lipid metabolism, RNA sequencing (RNA-seq) was performed on hepatic mRNA from mice treated with TM5614, a novel orally active small molecule inhibitor of PAI-1. Cluster analysis indicated homogeneity amongst samples in each group, with multiple changes in RNA expression across the genome (FIG. 3). Differential gene expression analysis revealed that PCKS9 was the most downregulated gene transcript in mice treated with TM5614 (86%, adjusted p=4.34×10⁻³², FIG. 4), whereas FGF 21 was the most upregulated transcript (568%, adjusted p=5.66×10⁻²⁸, FIG. 4). Gene ontology analysis confirmed that PAI-1 inhibition results in a coordinated and robust alteration in the expression of molecular factors responsible for lipid metabolism (FIG. 5). FIGS. 6-8 provide analysis of significant changes in mRNA in mice fed TM5614 in normal chow for 10 days as compared to mice not fed TM5614.

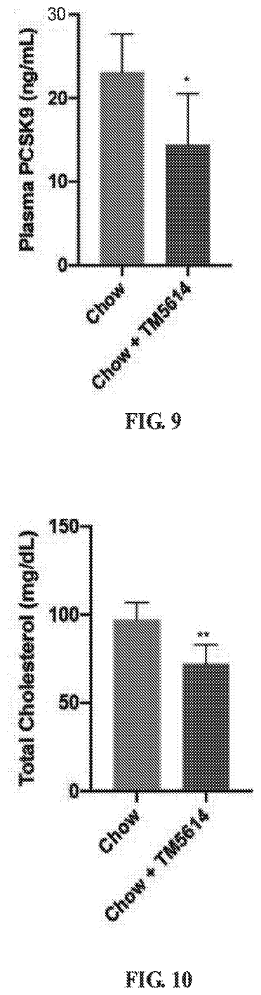

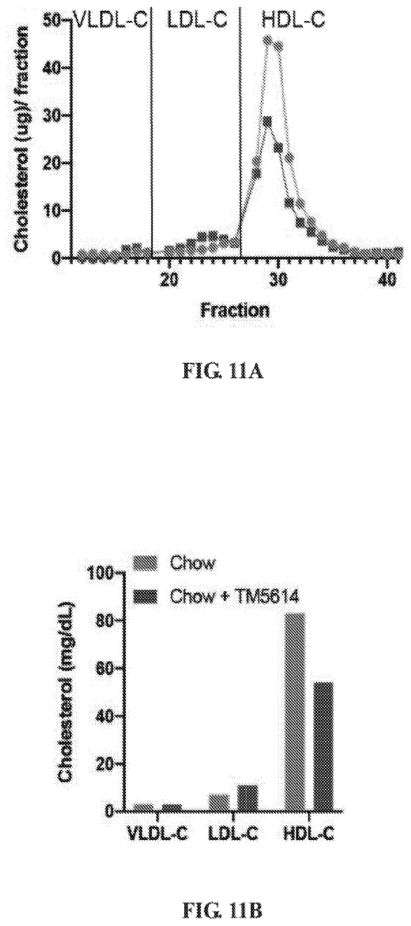

Consistent with these transcriptional changes, treatment with TM5614 led to a 40% decrease in plasma PCSK9 levels (FIG. 9, p<0.05) and this was accompanied by a 25% reduction in plasma cholesterol levels (FIG. 10, p<0.01).

### Example 3: TM5614 Reduces the HDL-Cholesterol Levels

The decrease in total cholesterol caused by PAI-1 inhibition was driven by a reduction in HDL cholesterol (HDL-C) level (FIG. 11A-B).

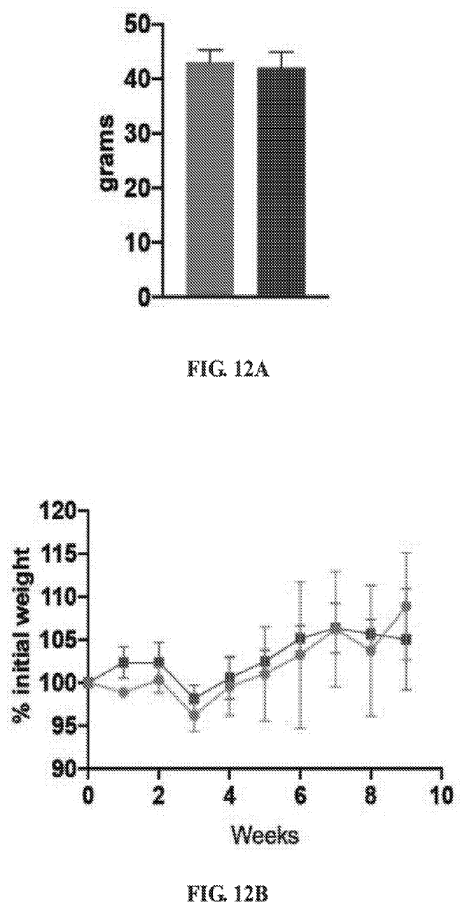

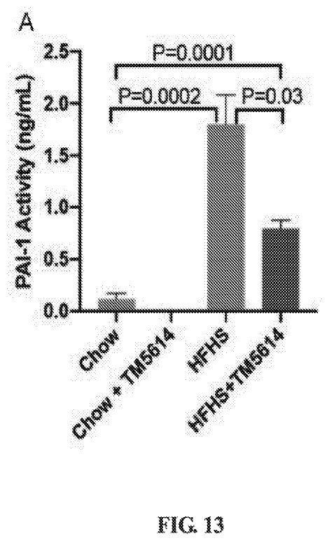

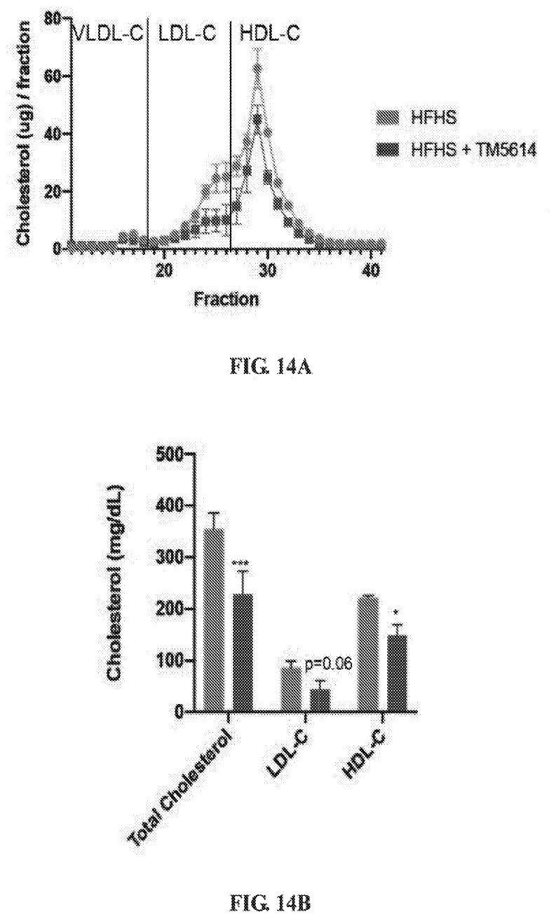

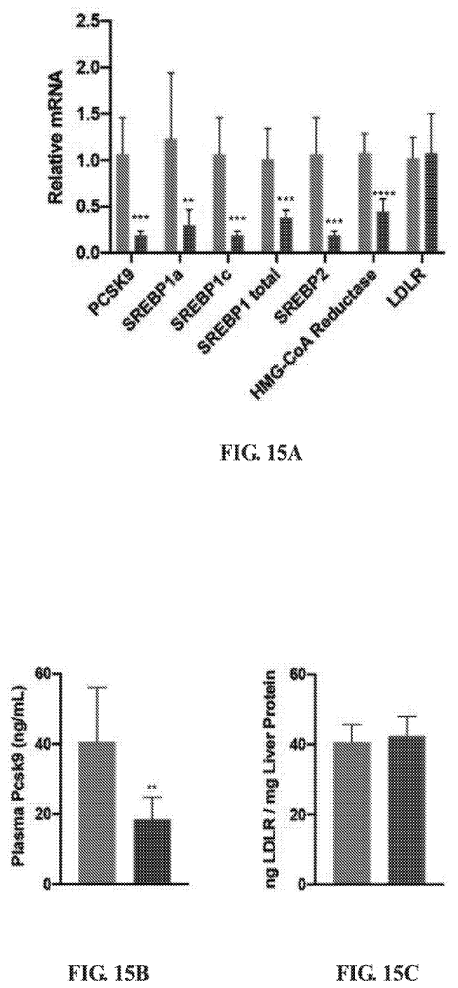

To evaluate the effects of PAI-1 inhibition on cholesterol metabolism, we used a high-fat, high-sugar (HFHS) diet to induce obesity and hyperlipidemia. WT C57BL/6J mice were placed on HFHS diet for 16 weeks, and then treatment with TM5614 was initiated in half of the animals for an additional 10 weeks, while the other half of the cohort remained on HFHS diet. The HFHS diet increased total cholesterol, LDL-C, and HDL-C, as expected. There were no differences between starting weights or weights during the second half of the trial between the experimental groups (FIG. 12A-12B). The HFHS diet produced a 16-fold increase in plasma PAI-1 activity and TM5614 administration resulted in a 56% reduction in PAI-1 activity (FIG. 13). Inhibition of PAI-1 led to a relative reduction of plasma total cholesterol by 37% (p<0.05), LDL-C by 48% (p=0.06), and HDL-C by 33% (p<0.05) (FIG. 14A-B).

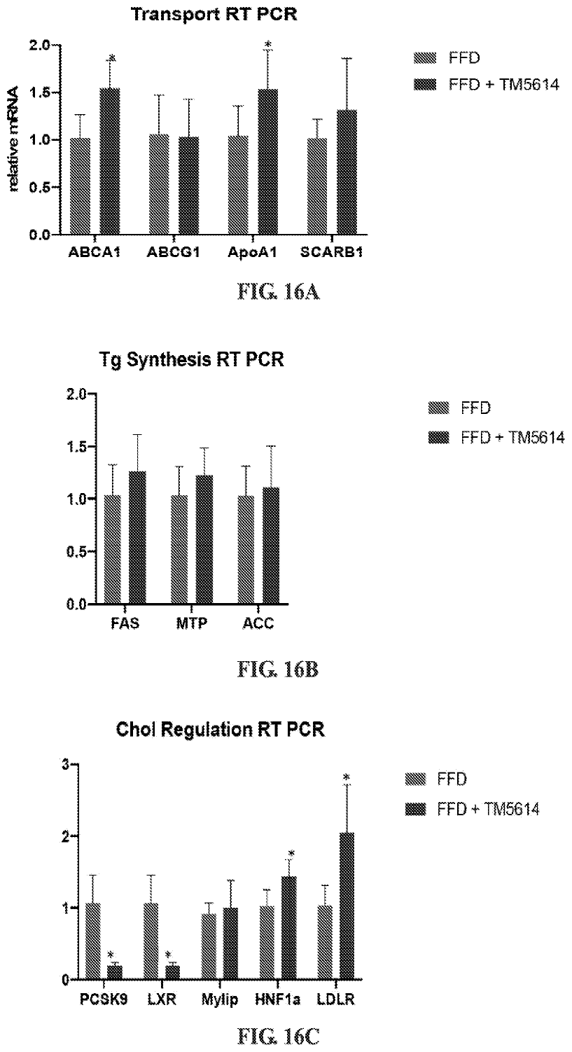

Hepatic PCSK9 mRNA expression was reduced by 81% in mice fed a HFHS supplemented with TM5614 (p<0.001). Other genes involved in lipid metabolism were also reduced (FIG. 15), including SREBP1a (70%, p<0.01), SREBP1c (81%, p<0.001), SREBP2 (42%, p<0.001), and HMG-CoA reductase (55%, p<0.0001). The reduction in PCSK9 mRNA expression was associated with a 55% reduction in the corresponding plasma levels of the protein (p<0.01, FIG. 15A), while LDLR mRNA and protein levels were unchanged (FIGS. 15B and 15C). FIG. 16A-C demonstrates RT-PCR quantification of mRNA changes with TM5614 in mice FFD fed mice. Most of the changes are in the cholesterol synthesis and regulation pathway.

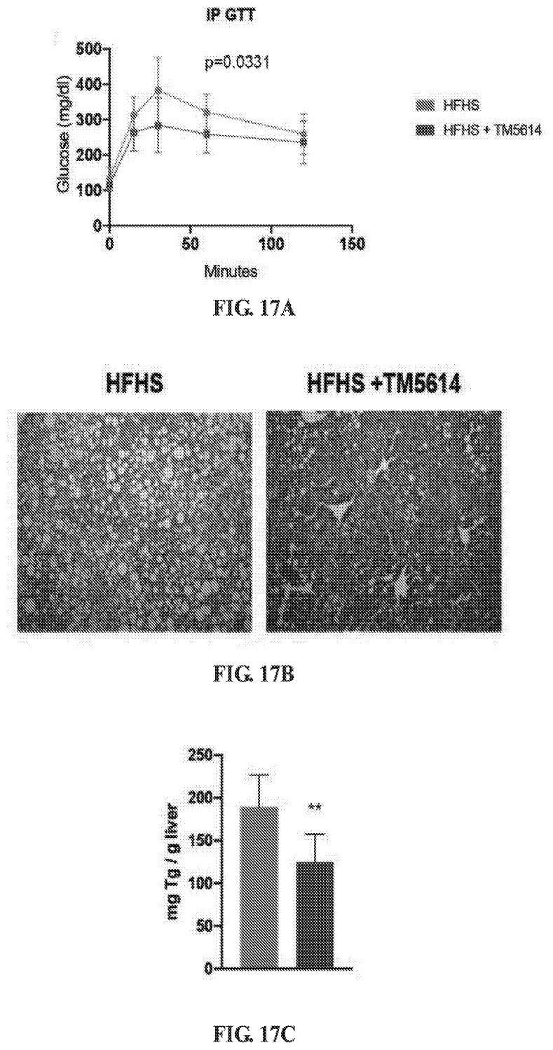

Consistent with other, inhibition with TM5614 was associated with preserved glucose tolerance and a reduction in hepatic steatosis (FIG. 17A-C).

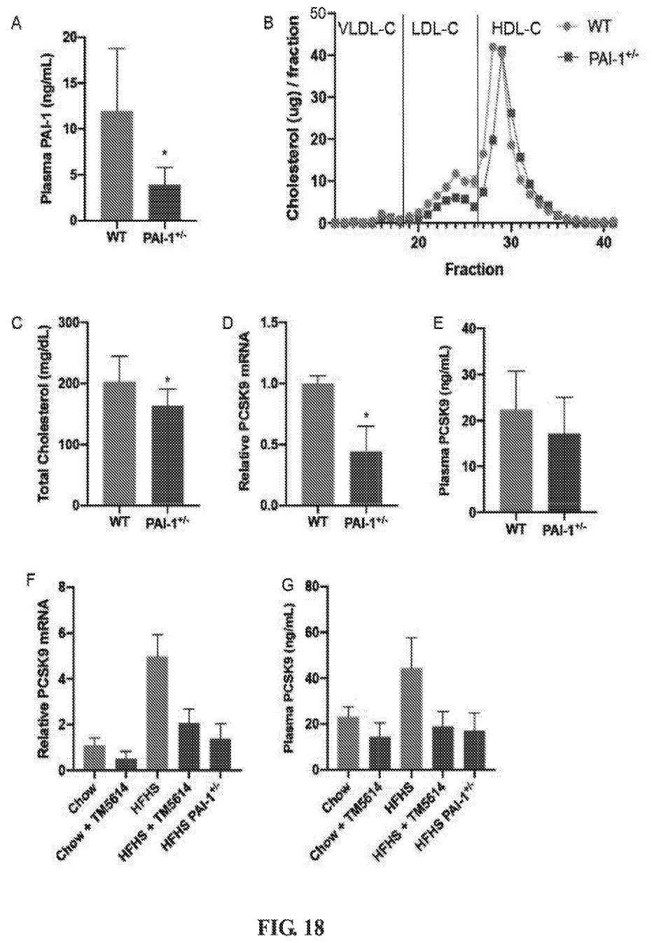

To confirm that the effects of TM5614 administration were due to specific inhibition of PAI-1 and not to off-target effects of the drug, we designed and performed similar studies in mice with partial PAI-1 deficiency (PAI-1^{+/-}) fed a HFHS diet for 16 weeks. These mice exhibit plasma PAI-1 levels that are approximately 50% of that seen in littermate controls (FIG. 18A), but comparable to the reduction observed in animals treated with TM5614. In these studies, PAI-1 deficiency was associated with a 20% lower plasma cholesterol level (p<0.05), driven by a decrease in LDL-C and HDL-C (FIG. 18B-C). Hepatic PCSK9 transcript levels were also reduced by 50% (p<0.05) (FIG. 18D), with a trend towards decreased plasma PCSK9 levels (FIG. 18E). A comparison of hepatic PCSK9 mRNA expression and plasma PCSK9 levels in all of the experimental groups described herein is shown in FIG. 18E-G. Taken together, these data suggest that PAI-1 directly influences the expression of PCSK9.

### Example 4: Relationship Between PAI-1 and PCSK9 in Plasma

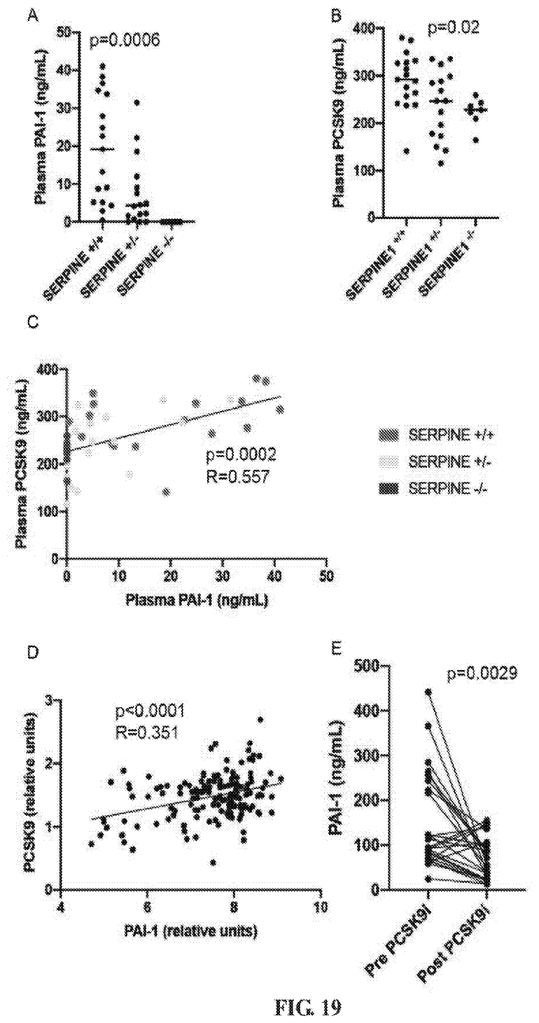

To determine if genetic deficiency in PAI-1 affects plasma PCSK9 levels in humans. PCSK9 levels were measured in frozen plasma samples previously obtained from members of the Indiana Swiss Amish community, which harbors a unique loss-of-function mutation in SERPINE1, the gene that codes for PAI-1. As previously reported, plasma PAI-1 levels are reduced by 50% in heterozygous carriers of the genetic variant and are essentially undetectable in the rare homozygous individuals (FIG. 19A, p<0.001). Individuals with either one (n=16, average age 45.1±18.9 years) or two copies (n=7, average age 27.3±5.9 years) of the genetic variant had significantly lower plasma PCSK9 levels compared to unaffected controls (n=17 average age 42.0±21.4 years) in the same cohort (FIG. 19B, p=0.02 by ANOVA). Furthermore, PCSK9 levels positively correlated with PAI-1 levels in all groups (FIG. 18 C, r=0.557, p<0.001).

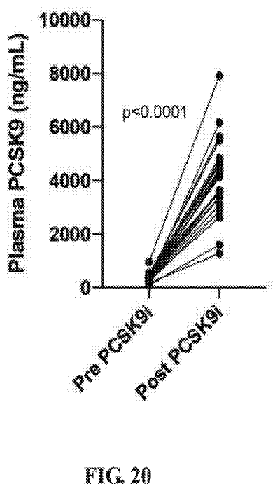

PAI-1 and PCSK9 levels were also quantified in plasma samples obtained from a human cohort with and without heart failure with preserved ejection fraction (HFpEF). There was again a positive correlation between PAI-1 and PCSK9 levels in these patients, similar to that in mice and in the Amish kindred (FIG. 19D, p<0.0001; R=0.351). Furthermore, we analyzed PAI-1 levels in a cohort of hypercholesterolemic patients and demonstrated that treatment with monoclonal antibodies blocking PCSK9 binding to the LDLR (evolocumab and alirocumab) produces a large increase in total plasma PCSK9 levels (FIG. 20) and significant reduction in plasma PAI-1 levels (FIG. 19E, p=0.0029). These data indicate that modulation of PCSK9 activity has a reciprocal effect on plasma PAI-1 levels.

### Example 5: PAI-1 Inhibition Reduces the Non-Glycosylated Form of PCSK9

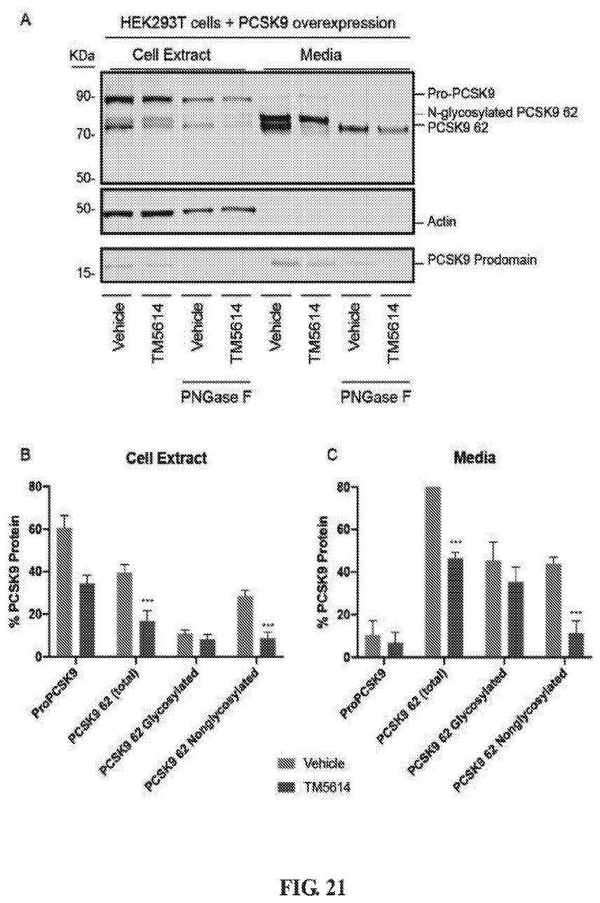

The mechanism by which PAI-1 affects PCSK9 homeostasis was determined by transfecting HEK293T cells with a human PCSK9 cDNA driven by a CMV promoter. Western blot analysis of the cell extract and media yielded three distinct PCSK9 forms, the pro-PCSK9 (72 kDa) and the glycosylated and non-glycosylated forms of mature PCKS9 (62 kDa). Treatment of cells with TM5614 reduced the accumulation of all PCSK9 forms with most robust reduction of the non-glycosylated form in both cell extracts (70±5%, p<0.001) and in conditioned media (75±7%, p<0.001) (FIG. 21A-B).

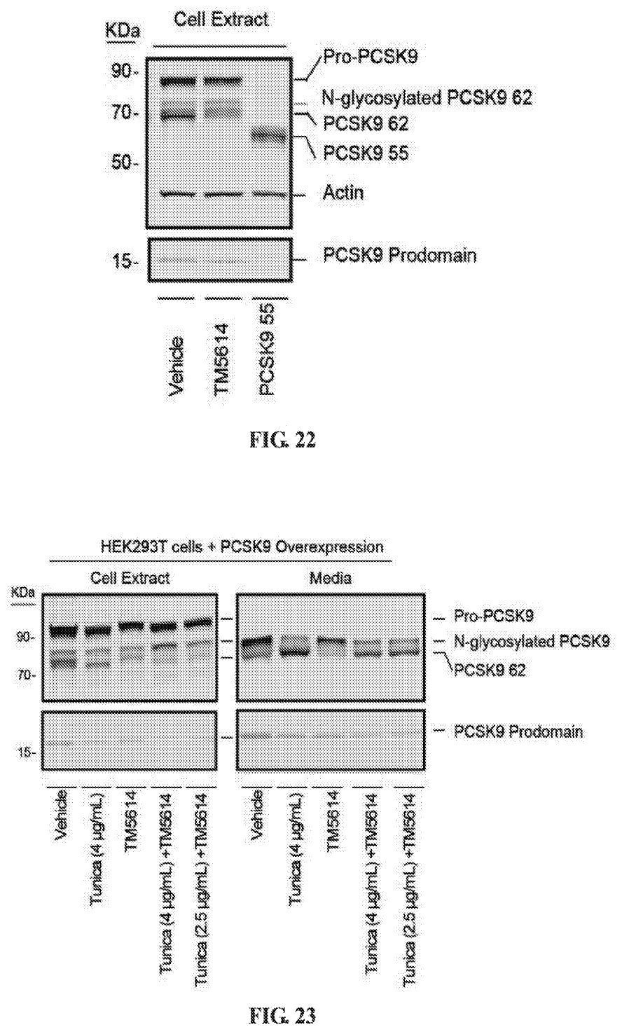

Apart from the PCSK9 forms described above, a distinct 55 kDa form of PCSK9 has been identified in plasma samples that is generated by furin-mediated proteolysis. Direct comparison of cells transfected with full length PCSK9 or a plasmid expressing the 55 kDa version of PCSK9 confirmed that the smaller band seen in cells transfected with PCSK9 is the non-glycosylated 62 kDa form and not the 55 kDa version of PCSK9 (FIG. 22). Treatment with PNGase F, an amidase that cleaves N-glycosylated products, confirmed that the two PCSK9 62 kDa bands represent glycosylated and non-glycosylated PCSK9 and shows that PAI-1 inhibition preferentially reduces the non-glycosylated version of PCSK9 (FIG. 21A-B). Additionally, the effects of TM5614 on the non-glycoslyated PCSK9 compete with the effects of tunicamycin, an inhibitor of N-glycosylation, suggesting that TM5614 has minimal effects on the glycosylated PCSK9 component (FIG. 23).

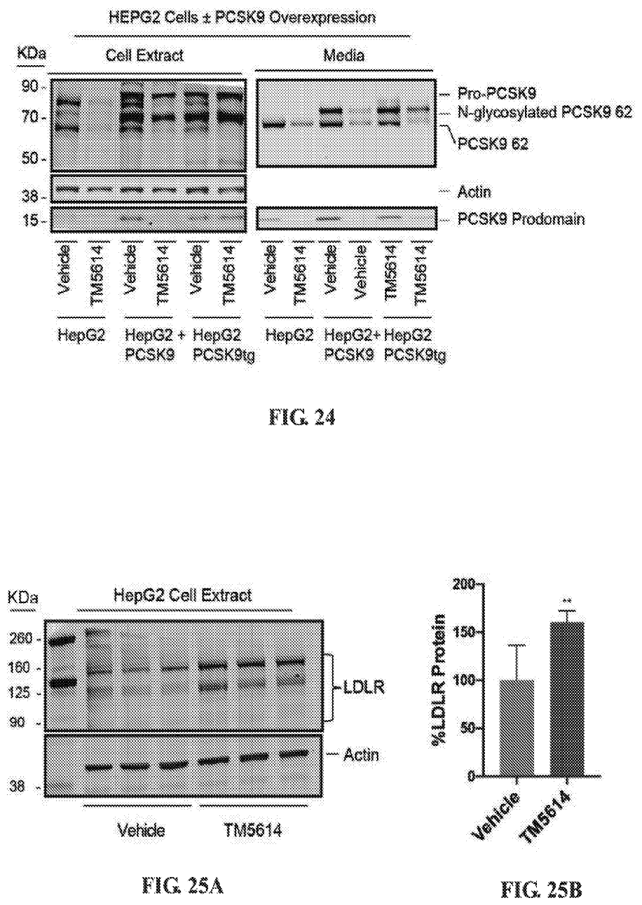

These experiments were repeated in WT HepG2 cells, FIGS. 24 and 25, (a human hepatoma line) and HepG2 cells that overexpress human PCSK9. Irrespective of the model used, treatment with TM5614 generated a reduction in PCSK9 (FIG. 23, 57±20%; (p<0.001) with selective reduction in the non-glycosylated form. In addition, HepG2 cells treated with TM5614 exhibited an increase in LDLR (65±7%, p<0.001), suggesting that PAI-1 regulation of PCSK9 levels has an impact in cholesterol clearance mediated by increased LDLR levels (FIG. 20C).

### Example 6: In Vitro Assay Demonstrating PCSK9 can be Cleaved by uPA

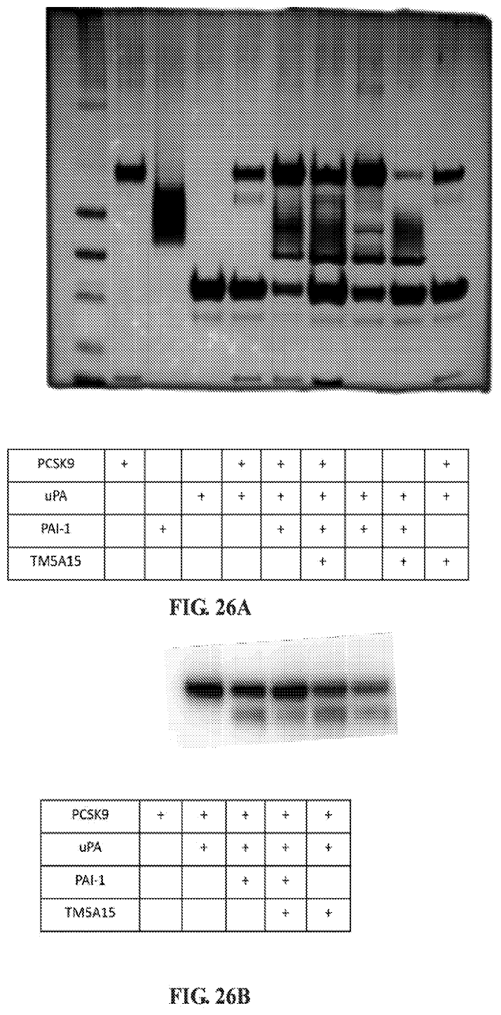

Referring now to FIG. 26A-B, in vitro recombinant human low molecular weight urokinase efficiently promoted the proteolytic degradation of recombinant human PCSK9. This reaction was inhibited by the presence of recombinant human PAI-1 and restored by the addition of a small molecule PAI-1 inhibitor. FIG. 26A is a silver stain, and 26B a Western Blot for PCSK9.

As stated above, while the present application has been illustrated by the description of embodiments, and while the embodiments have been described in considerable detail, it is not the intention to restrict or in any way limit the scope of the appended claims to such detail. Additional advantages and modifications will readily appear to those skilled in the art, having the benefit of this application. Therefore, the application, in its broader aspects, is not limited to the specific details and illustrative examples shown. Departures may be made from such details and examples without departing from the spirit or scope of the general inventive concept.

1. A method for reducing plasma LDL, HDL, cholesterol, or a combination thereof in a subject, the method comprising orally administering to the subject a therapeutically effective amount of a plasminogen activator inhibitor 1 (PAI-1) inhibitor,
wherein the treatment with the plasminogen activator inhibitor 1 (PAI-1) inhibitor decreases plasma LDL, HDL, cholesterol, or a combination thereof in a subject by about 25% to about 40% when compared to plasma LDL, HDL, cholesterol, or a combination thereof in a untreated subject.

2. The method of claim 1, the inhibitor of plasminogen activator inhibitor 1 (PAI-1) is TM5614, TM5A15, or a combination thereof.

3. The method of claim 1, further comprising co-administration to the subject of a statin and plasminogen activator inhibitor 1 (PAI-1) inhibitor.

4. The method of claim 1, further comprising co-administration to the subject of a PCSK9 inhibitor and plasminogen activator inhibitor 1 (PAI-1) inhibitor.

5. The method of claim 1, wherein the plasminogen activator inhibitor 1 (PAI-1) inhibitor is administered in a dosage of about 20 mg/kg.

6. The method of claim 1, wherein the plasminogen activator inhibitor 1 (PAI-1) inhibitor is administered to the subject daily.

7. A method of treating a metabolic disorder in a subject comprising:
administering to the subject an inhibitor of plasminogen activator inhibitor 1 (PAI-1),
wherein treatment with the plasminogen activator inhibitor 1 (PAI-1) inhibitor changes at least one metabolic measurement of the patient selected from the group consisting of: fasting glucose level, fasting plasma insulin level, fasting plasma LDL, plasma cholesterol level, plasma HDL, plasma PCSK9 level, plasma plasminogen activator inhibitor 1 (PAI-1) activity, or a combination thereof, when compared to the metabolic measurement of a subject in an untreated condition.

8. The method of claim 7, the inhibitor of plasminogen activator inhibitor 1 (PAI-1) is TM5614, TM5A15, or a combination thereof.

9. The method of claim 7, wherein the inhibitor of plasminogen activator inhibitor 1 (PAI-1) is administered orally.

10. The method of claim 7, the metabolic disorder comprising obesity, type 2 diabetes mellitus, nonalcoholic fatty liver disease, non-alcoholic steatohepatitis, hyperlipidemia, metabolic syndrome, and cardiovascular disease, hypercholesterolemia, familial hypercholesterolemia, elevated LDL, HDL, or a combination thereof.

11. The method of claim 7, the plasminogen activator inhibitor 1 (PAI-1) inhibitor decreases plasma cholesterol by about 25% to about 40%.

12. The method of claim 7, the plasminogen activator inhibitor 1 (PAI-1) inhibitor decreases HDL-C by about 30%.

13. The method of claim 7, the plasminogen activator inhibitor 1 (PAI-1) inhibitor decreases LDL-C by about 50%.

14. The method of claim 7, the plasminogen activator inhibitor 1 (PAI-1) inhibitor reducing PAI-1 activity by about 50%.

15. The method of claim 7, the plasminogen activator inhibitor 1 (PAI-1) inhibitor reducing hepatic PCSK9 mRNA expression by about 80%, thereby reducing plasma PCSK9 protein.

16. The method of claim 7, the plasminogen activator inhibitor 1 (PAI-1) inhibitor effectively reducing plasma PCSK9 protein by about 25% to about 50%.

17. The method of claim 7, the plasminogen activator inhibitor 1 (PAI-1) inhibitor effectively reducing plasma non-glycosylated PCSK9 protein.

18. The method of claim 7, further comprising co-administration to the subject of a statin and plasminogen activator inhibitor 1 (PAI-1) inhibitor.
